(19) United States
(12) Patent Application Publication
(10) Pub. No.: US 2017/0049969 A1
(43) Pub. Date: Feb. 23, 2017

(54) INDICATOR

(71) Applicant: New Injection Systems Ltd, Ipswich, Suffolk (GB)

(72) Inventors: Stephen Dunne, Ipswich, Suffolk (GB); Tom Oakley, Cambridge, Cambridgeshire (GB); Douglas Arthur Emmott, Woodbridge, Suffolk (GB)

(21) Appl. No.: 15/307,886

(22) PCT Filed: May 1, 2015
(86) PCT No.: PCT/GB2015/051304

§ 371 Date: Oct. 31, 2016

(30) Foreign Application Priority Data
May 1, 2014 (GB) 1407680.6
Feb. 12, 2015 (GB) PCT/GB2015/050393

(51) Int. Cl. A61M 5/315; A61M 5/20; A61M 5/24

(52) U.S. Cl. CPC A61M 5/31591; A61M 5/24; A61M 5/2033; A61M 5/2466; A61M 5/315; A61M 2005/3126

(57) ABSTRACT

An auto-injector for delivering a liquid medicament comprises: a collapsible container for containing the liquid medicament (26); an openable closure; and an elongate indication member (21) located at least partially within the rigid outer container (22) and arranged to move longitudinally towards the distal end of the collapsible container when the openable closure is opened, in which longitudinal movement of the elongate indication member (21) produces visual, and/or audio and/or tactile feedback relating to injection progress to a user of the auto-injector.

The invention relates to an auto-injector for delivering a liquid medicament, which comprises an elongate indication member. Longitudinal movement of this elongate indication member provides visual and/or audio and/or tactile feedback relating to injection progress to a user of the auto-injector. The invention also relates to a method of using such an auto-injector.

### BACKGROUND

An auto-injector is a common type of injector which is designed to deliver a single dose of a liquid medicament. Auto-injectors provide automation of the injection stroke, which alleviates the need for a patient or medical professional to actuate a plunger rod to deliver medication. Many auto-injectors are types of spring-loaded syringes.

Some auto-injectors provide visual indication to confirm that the full dose of a liquid medicament has been delivered. However, unlike conventional syringes, prior art auto-injectors do not allow a user to monitor the release of a liquid medicament from the auto-injector during injection.

### DESCRIPTION OF THE INVENTION

The invention provides an auto-injector and a method of using an auto-injector, as defined in the appended independent claims, to which reference should now be made. Preferred or advantageous features of the invention are set out in dependent sub-claims.

Thus, an auto-injector for delivering a liquid medicament may comprise a rigid outer container; a collapsible container for containing the liquid medicament located within the rigid outer container, the collapsible container having a proximal end and a distal end; an openable closure coupled to the distal end of the collapsible container for retaining the liquid medicament within the collapsible container; an injection means coupled to the openable closure for delivering the liquid medicament from the collapsible container; and pressurising means located within the rigid outer container; the auto-injector further comprising: an elongate indication member. The pressurising means may be located within the rigid outer container, the pressurising means providing a force acting on the proximal end of the collapsible container that urges the proximal end of the collapsible container towards the distal end of the collapsible container, thereby pressurising the liquid medicament contained within the collapsible container such that the liquid medicament is delivered through the injection means when the openable closure is opened. The elongate indication member may be located at least partially within the rigid outer container and arranged to move longitudinally towards the distal end of the collapsible container when the openable closure is opened, in which longitudinal movement of the elongate indication member produces visual and/or audio and/or tactile feedback relating to injection progress to a user of the auto-injector. That is, the feedback may be visual feedback, audio feedback, tactile feedback, or feedback comprising any two or more of visual feedback, audio feedback, and tactile feedback.

An advantage of this auto-injector over prior art auto-injectors is that the production of visual and/or audio and/or tactile feedback allows a user to monitor the injection progress. For example, the elongate indication member may inform the user when the injection process has begun and/or when the injection process has terminated and/or how far the injection process has proceeded after injection initiation.

The incorporation of indication members into auto-injectors that do not pre-pressurise a medicament may pose significant challenges due to the high complexity of such devices. By contrast, an indication member may be advantageously incorporated into pressurised auto-injectors using relatively simple means from a manufacturing perspective. Thus, an auto-injector comprising a pre-pressurised medicament may allow incorporation of a cost effective indicator for indicating progress of an injection. Moreover, the coupling of longitudinal movement of the elongate indication member to the production of visual and/or audio and/or tactile feedback provides a cost-effective solution to the problem of providing auto-injector users with an indication of injection progress.

The collapsible container may be defined in part by rigid internal walls and in part by a piston that is slideably arranged within the rigid internal walls, such that the volume of the collapsible container varies depending on the position of the piston. The collapsible container may be a substantially cylindrical chamber. For example, the collapsible container may be a standard glass or plastic pre-filled syringe or cartridge or glass container containing a piston.

The collapsible container may be a bellows coupled to an openable closure. The collapsible container may include both a bellows and a piston, for example a bellows may provide a lining to a collapsible container defined by rigid walls and a piston.

A distal end of the elongate indication member may be connected, directly or indirectly, to a proximal end of the collapsible container. For example, in embodiments in which the auto-injector comprises a piston, the elongate indication member may be connected to the piston such that longitudinal movement of the piston towards a distal end of the collapsible container upon opening of the openable closure causes longitudinal movement of the elongate indication member. Alternatively, the elongate indication member may be connected to the end of a bellows, which moves longitudinally towards a distal end of the collapsible container upon opening of the openable closure. Alternatively, the elongate indication member may be connected to a spring, which moves longitudinally towards a distal end of the collapsible container upon opening of the openable closure.

The openable closure may be a normally-closed valve, such as an aerosol valve. Such valves are well known and understood. The aerosol valve may be a springless valve in order to minimise the number of components or materials in contact with the liquid medicament and reduce the risk of unwanted drug/material interactions. Alternatively, the aerosol-valve may comprise a polymeric spring so that a metal is not arranged in contact with the liquid medicament during storage. In one embodiment, the entire openable closure is formed entirely of polymeric materials. Opening of the normally-closed valve may allow passage of the liquid medicament from the collapsible container.

In some embodiments the normally-closed valve may comprise a pierceable septum and a piercing means. The septum may be pierced by a hollow needle or cannula that is in fluid communication with a delivery means such as a hypodermic needle. When the septum is pierced the force applied to the collapsible container may force the liquid medicament out of the collapsible container.

The auto-injector may be a hand-held auto-injector.

A portion of the elongate indication member may extend beyond the rigid outer container, thereby providing a visual indication of injection progress to a user of the auto-injector. Such a portion, herein described as an indicator portion, may be designed to be easily visible to a user of the injector. For example, the indicator portion may have a larger diameter than the rest of the elongate indication member, and/or it may be brightly coloured. In one embodiment, the indicator portion may be disc-shaped. Additionally or alternatively, the indicator portion may comprise two or more segments having contrasting colours, such as green and red. These contrasting colours allow movement (e.g. rotation) of the indicator portion to be readily observed by a user of the auto-injector.

The rigid outer container may comprise a window through which a user of the auto-injector may monitor the movement of the elongate indication member, thereby providing feedback on the injection progress to a user of the auto-injector.

The window may be a transparent window, thereby allowing visual monitoring of injection progress by a user of the auto-injector. Alternatively, the auto-injector may comprise a transparent cap or a cap having a window (e.g. a transparent window) through which a user of the auto-injector may observe the movement (e.g. longitudinal or rotational movement) of the elongate indication member, thereby providing a visual indication of injection progress to a user of the auto-injector. In one embodiment, the auto-injector may be configured to allow viewing of the rotation of an indicator portion (as described above) through the viewing window.

Alternatively, the rigid outer container may comprise a finger window through which a user of the auto-injector may insert a finger in order to monitor the movement (e.g. longitudinal movement) of the elongate indication member, thereby providing tactile indication of injection progress to a user of the auto-injector. For example, the elongate indication member may comprise an indicator portion at its proximal end, which has a larger diameter than the non-indicator portion of the elongate indication member. Prior to injection, the distance between the indicator portion and the distal end of the collapsible container may be greater than the distance between the finger window and the distal end of the collapsible container. Prior to injection, the indicator portion would not be felt in the finger window. During injection, the elongate indication member moves longitudinally towards a distal end of the auto-injector until it can be felt through the finger window. The finger window may be substantially semi-circular in shape.

The auto-injector may be configured such that prior to injection the end of the elongate indication member protrudes beyond the proximal end of the rigid outer container. After initiation of injection, the end of the elongate indication member may no longer protrude beyond the proximal end of the rigid outer container. The protrusion of the elongate indication member beyond the proximal end of the rigid outer container may be detectable by touch and/or sight.

Longitudinal movement of the elongate indication member may produce audio feedback in the form of a series of clicks. In one embodiment, the feedback is not in the form of a series of clicks.

The elongate indication member may be connected to an indicator portion, wherein longitudinal movement of the elongate indication member is translated into rotational movement of the indicator portion relative to the auto-injector casing and said rotational movement of the indicator portion provides visual, audio and/or tactile feedback.

The elongate indication member may have a shaped cross-section which twists along the length of a portion of the elongate indication member and the indicator portion may have a slot that is shaped to engage with the elongate indication member such that longitudinal movement of the elongate indication member is translated into rotational movement of the indicator portion relative to the auto-injector casing. The shape of the elongate indication member may be produced by moulding. The indication portion may comprise projections such as teeth designed to engage with a sprung member, such as a sprung projection, to form an audible indication in the form of clicks. The indicator portion may comprise ratchet means, such as ratchet teeth and a ratchet tang, to ensure that the indicator portion only rotates in one direction. A ratchet means may additionally provide an audible indication of the progress of injection in the form of clicks.

The elongate indication member may be cylindrical with a grooved thread and the indicator portion may be configured to engage with the grooved thread such that longitudinal movement of the elongate indication member is translated into rotational movement of the indicator portion relative to the auto-injector casing. For example, the indicator portion may comprise a pin, which engages with the grooved thread. The grooved thread may be produced by moulding.

The rigid outer container may comprise a finger window through which a user of the auto-injector may insert a finger in order to monitor the movement (e.g. rotational movement) of the indicator portion, thereby providing tactile indication of injection progress to a user of the auto-injector.

The elongate indication member may provide visual, audio and/or tactile feedback by mechanical or electronic means. Advantages of producing visual, audio and/or tactile feedback by mechanical means include simplicity, reduced cost and fewer regulatory standards to comply with, whereas advantages of producing visual, audio and/or tactile feedback by electronic means include flexibility in terms of the feedback generated (including the generation of a variety of sounds, flashing lights and/or colours). In addition, electronic indication means may indicate an end-point before the injection end and they may provide additional monitoring possibilities.

The elongate indication member may comprise a sounder which provides audio feedback upon movement of the elongate indication member; and/or a light, which provides visual feedback upon movement of the elongate indication member.

The elongate indication member may be adapted to close electrical contacts as it moves longitudinally towards the distal end of the collapsible container, closure of the electrical contacts providing feedback relating to initiation and injection progress to a user of the auto-injector. For example, the auto-injector may comprise electrical contacts, which are open before and after injection, and an elongate indication member having a central portion which is wider in diameter than the end portions on either side of the central portion. In this embodiment, the electrical contacts may be closed by the central portion of the elongate indication member as it moves longitudinally towards the distal end of the collapsible container, and the contacts may re-open at the end of injection when the narrow end portion of the elongate indication member reaches the electrical contacts.

The elongate indication member may comprise a battery and a printed circuit board (PCB). In addition, the elongate indication member may comprise a sounder and/or a light. The battery and the sounder and/or light may be connected to the PCB by pressed metal spring contacts. The advantage of pressed metal spring contacts is that they are cheaper than soldering wires or using connectors.

The contacts may be placed within or outside of the rigid container. The contacts may be placed within the elongated indication member. For example, the contacts may be positioned outside of the rigid container, but within the elongated indication member.

The pressurising means may be a biasing means, such as a spring. The spring may be a helical spring. In auto-injectors comprising a spring and a piston, the elongate indication member may comprise a flange at its distal end, which protects the piston from being damaged by the pressure of the spring.

The pressurising means may apply a continuous pressure to the collapsible container such that the liquid medicament is maintained under pressure during storage. There are a number of benefits provided by storage of a liquid medicament under a continuous pressure. For example, storage of a liquid medicament under pressure advantageously improves leak detection. If the primary container or a closure of the container is damaged, the positive storage pressure causes the medicament to leak thereby alerting the user to a problem. The positive pressure in the container during storage also minimizes the risk of drug contamination and loss of sterility.

The auto-injector may comprise pressurising means arranged to apply a first force to the collapsible container to continuously pressurise a liquid medicament contained therein during storage of the liquid medicament. Such pressurising means may be actuatable to apply a second force to the collapsible container, the second force being greater than the first force.

The fact that the pressurising means can apply a second force to the collapsible container, which is greater than the first force, allows the collapsible container to be formed from fragile materials such as glass yet still be used for high pressure delivery of liquid medicaments. High pressure delivery is useful for reducing the delivery time required for viscous medicaments, such as biological medicaments, whilst still allowing small gauge needles to be used. As a low storage pressure is used, the benefits of storing the medicament under positive pressure may be achieved. The hazards of pressurising a fragile glass container and storing and shipping a highly pressurised container may be avoided. As a relatively high delivery pressure can subsequently be applied to the collapsible container when required, the benefits of high pressure delivery may be achieved, which may be particularly advantageous in the context of delivering highly viscous medicaments. The second higher force need not be applied to the collapsible container for any significant length of time, thereby reducing hazards associated with pressurising the container.

The pressurising means may comprise a first biasing means and a second biasing means. Thus, the first force may be exerted by the first biasing means and the second force may be exerted by the second biasing means. It may be preferred that the first biasing means is a first spring, for example a first helical spring, and/or the second biasing means is a second spring, for example a second helical spring. Other biasing means may be envisaged. For example, the first force and/or the second force may be provided by gas pressure.

The auto-injector may comprise a housing locating the collapsible container and the pressurising means, and the pressurising means may be retained within the housing such that it acts on the collapsible container. Thus, in some embodiments a first biasing means may be retained or located within the housing such that it continually acts on the collapsible container until and/or after the pressurising means is actuated. A second biasing means may be retained or located within the housing such that it acts on the collapsible container after the pressurising means has been actuated. The first biasing means and the second biasing means may be arranged within the housing in parallel or in series. If the first and second biasing means are springs, it is preferred that the second biasing means is a spring having a higher compression force than the first biasing means during its unactuated condition.

Preferably, when the pressurising means is in an unactuated condition the second biasing means is restrained within the auto-injector such that it does not exert a force on the collapsible container. Actuation of the pressurising means may then cause the second biasing means to exert a force resulting in the second force being applied to the collapsible container.

In embodiments in which the pressurising means is arranged to apply a storage force and a higher delivery force, the pressurising means may comprise only a single biasing means arranged to apply the first force and the second force, the auto-injector being arranged such that both the first force and the second force can be exerted by the single biasing means. For example, the single biasing means may be a single helical spring. For example, when the pressurising means is in an unactuated condition a first portion of the single biasing means may be restrained within the auto-injector such that only a second portion of the single biasing means exerts the first force on the collapsible container. Actuation of the pressurising means may then allow both first and second portions of the single biasing means to exert the second force on the collapsible container. If the single biasing means is a helical spring having, for example, eight coils, the spring may be restrained by a restraining means such that only one or two coils of the spring act on the collapsible container. The one or two coils of the spring exert the first force. Actuating the pressurising means by removal of the restraining means allows all eight of the coils of the spring act on the collapsible container thereby exerting the second force that is greater than the first force.

The pressurising means may be actuated to apply the second force to the collapsible container automatically or manually. For example, the pressurising means may be actuated to apply the second force to the collapsible container by depressing a button. The button may automatically actuate the pressurising means when the injector is used. Alternatively, the pressurising means may be actuated by removing a cap or a latch. The elongate indication member may comprise the latch.

The auto-injector may comprise a releasable retaining means which can releasably retain the pressurising means. The releasable retaining means may be part of the elongate indication member. The pressurising means may be actuated by release of the retaining means. The releasable retaining means may comprise a retaining arm, which at one end attaches to the pressurising means and at the other end releasably attaches to the auto-injector, for example to a cap at the proximal end of the auto-injector. In one embodiment, the releasable retaining means comprises a catch, e.g. a hook, that is able to releasably engage with the cap, wherein the cap comprises a hole that allows passage of the catch upon displacement of the hook from the cap. In one embodiment, the elongate indication member comprises a screw thread and the auto-injector comprises a nut that is engageable with the screw thread, wherein the pressurising means is actuated to apply the second force to the collapsible container by rotating the nut until the nut disengages the screw thread. For example, rotation of the nut by 45 to 360 degrees around the screw thread may actuate the pressurising means to apply the second force to the collapsible container.

An auto-injector according to any embodiment may comprise a liquid medicament, for example a liquid biological medicament. That is, the auto-injector may be pre-filled with a liquid medicament contained within the collapsible container. The auto-injector may be transported and stored with a liquid medicament within the collapsible container. In embodiments of auto-injectors applying a first force and a second force, the first force may act to continuously pressurise the liquid medicament to a pressure that is greater than atmospheric pressure but lower than 10 bars, preferably less than 5 bars. The pressure in the liquid medicament may be less than 10 bars, 9 bars, 8 bars, 7 bars, 6 bars, 5 bars, 4 bars, 3 bars, or 2 bars when the first force is applied to the collapsible container.

The second force may act to continuously pressurise the liquid medicament to a pressure that is greater than the first pressure and higher than 5 bars, preferably higher than 10 bars. The pressure in the liquid medicament may be higher than 6 bars, 7 bars, 8 bars, 9 bars, 10 bars, 12 bars, 15 bars, or 20 bars when the second force is applied to the collapsible container. The fact that the pressure in the liquid medicament during storage is lower relative to a delivery pressure reduces the stresses in the auto-injector, thereby increasing the safety and shelf-life of the auto-injector.

The auto-injector may comprise a glass container housing a piston and a first spring and a second spring arranged to act on the piston. A storage pressure may be applied to the piston by the first spring and a delivery force may be provided to the piston by the second spring. The auto-injector may include a locking and release mechanism for the second spring or main power spring. In this way during storage the collapsible container is kept at a lower than injection pressure to minimize stresses in the auto-injector.

In any embodiment described herein, the auto-injector may comprise means for establishing fluid communication between the collapsible container and the injection means, such that the pressurised liquid medicament is automatically delivered through the injection means when communication has been established. The injection means may be a needle, e.g. a hypodermic needle. Alternatively, the auto-injector may be a needleless auto-injector, e.g. using a needleless transdermal delivery system.

The auto-injector may further comprise a shield for the injection means, for example a needle shield. A linkage system may be provided, wherein said linkage system automatically actuates the pressurising means when the shield is retracted or removed. Thus, the auto-injector may be made ready for delivery when the shield is retracted or removed.

The auto-injector may comprise: a rigid outer container; a medicament container defining a substantially cylindrical chamber containing a liquid medicament, wherein the medicament container is located within the rigid outer container, a proximal end of the chamber being closed by a piston slidably located within the cylindrical chamber, and a distal end of the chamber being closed by a container seal spanning an opening at a distal end of the medicament container; biasing means coupled to the piston and acting to bias the piston towards the container seal, thereby pressurising the liquid medicament; a hypodermic needle for parenteral administration of the liquid medicament; a removable needle cap for maintaining the hypodermic needle in sterile conditions until use; means for establishing fluid communication between the chamber and the hypodermic needle such that the pressurised liquid medicament is automatically delivered through the hypodermic needle when communication has been established; and an elongate indication member located at least partially within the rigid outer container and arranged to move longitudinally towards the distal end of the medicament container when fluid communication has been established, in which longitudinal movement of the elongate indication member produces visual, and/or audio and/or tactile feedback relating to injection progress to a user of the auto-injector.

In one embodiment, the container seal is a pierceable septum spanning the opening at the distal end of the container and the means for establishing fluid communication is a valve comprising a valve housing defining a bore, the valve housing coupled to the distal end of the container such that the pierceable septum is located at a proximal end of the bore, and a shuttle slidably retained within the bore, the shuttle comprising a piercing element for piercing the pierceable septum when the shuttle is moved towards the proximal end of the bore to establish fluid communication between the chamber and the hypodermic needle. The piercing element and the hypodermic needle may be formed by opposite ends of a double-ended needle located by the shuttle.

The auto-injector may comprise: a medicament container defining a substantially cylindrical chamber containing a liquid medicament, a proximal end of the chamber being closed by a piston slidably located within the cylindrical chamber, and a distal end of the chamber being closed by a container seal spanning an opening at a distal end of the medicament container; pressurising means arranged to apply a first force to the piston and acting to bias the piston towards the container seal, thereby continuously pressurising the liquid medicament during storage of the liquid medicament, wherein the pressurising means is actuatable to apply a second force to the collapsible container, the second force being greater than the first force; a hypodermic needle for parenteral administration of the liquid medicament, a removable needle cap for maintaining the hypodermic needle in sterile conditions until use, and means for establishing fluid communication between the chamber and the hypodermic needle such that the pressurised liquid medicament is automatically delivered through the hypodermic needle when communication has been established.

A method of using an auto-injector comprising a collapsible container containing a liquid medicament; and pressurising means arranged to apply a first force to the collapsible container to continuously pressurise a liquid medicament contained therein during storage may comprise the steps of: (a) actuating the pressurising means to apply a second force to the collapsible container, the second force being greater than the first force, and (b) establishing communication between the collapsible container and a delivery means for delivering the liquid medicament to the patient, the second force acting to eject the liquid medicament from the collapsible container through the delivery means.

The pressurising means may be actuated simultaneously with or before communication is established between the collapsible container and the delivery means. Alternatively, communication may be established between the collapsible container and the delivery means before the pressurising means is actuated. The auto-injector may be any auto-injector described above.

A method of using an auto-injector as defined above may comprise the steps of: (a) opening the openable closure to allow delivery of the liquid medicament through the injection means and longitudinal movement of the elongate indication member towards the distal end of the collapsible container; and (b) monitoring the injection progress via visual, and/or audio and/or tactile feedback produced by the longitudinal movement of the elongate indication member. The method may comprise the step of establishing communication between the collapsible container and the delivery means for delivering the liquid medicament to a patient.

The present invention may be used in conjunction with any drug whether a solution or a suspension or a mixture of these of any viscosity and density. Any of the drugs listed below, either on its own or a mixture thereof, may be injected using an auto-injector as disclosed herein:

17-alpha hydroxyprogesterone caproate, Corticotropin (ACTH), Laronidase, Factor VIII, Von Willebrand Factor Complex, Alefacept, Apomorphine Hydrochloride, Darbepoetin Alfa, Nelarabine, Bevacizumab, Interferon beta-1a, 11 mcg, Interferon beta-1a, 33 mcg, Factor IX complex, Interferon beta-1b, Ibandronate Sodium, Botulinum Toxin, Protein C Concentrate, Alglucerase, Imiglucerase, Injection, Secretin, Synthetic, Human, 1 Microgram, Glatiramer actate, Decitabine, Desmopressin acetate, Idursulfase, Etanercept, Epoetin alfa, Anadalufungin, Cetuximab, Ethanolamine Oleate, Hyaluronic acid derivatives, Agalsidase beta, Factor IX non-recombinant, Factor IX recombinant, Factor VIII (human), Factor VIII (porcine), Factor VIII recombinant, Feiba VH, Immune globulin (intravenous) (IVIG), Enfuvirtide, Immune globulin (intravenous) (IVIG), Somatropin, Hepatitis B Immune, Globulin (intravenous) (IVIG), Trastuzumab, von Willebrand factor complex, Adalimumab, Insulin for administration through DME (i.e., insulin pump), Hyaluronic acid derivatives, Mecasermin, Gefitinib, Levoleucovorin calcium, Ranibizumab Injection, Pegaptnib, Urofollitropin, Micafungin, Botulinum toxin type B, Aglucosidase alfa, Galsulfase, Somatropin, Factor Vila, Atacept, Hyaluronic acid derivatives, Hyaluronan derivative, Immune globulin (intravenous) (IVIG), Hemin, Peginterferon alfa-2a, Peginterferon alfa-2b, Epoetin alfa, Somatrem, Efalizumab, Interferon beta-1a, subq, Zoledronic Acid, Infliximab, Treprostinil, Fluocinolone acetonide, intravitreal implant, Zidovudine, Eculizumab, Lanreotide, Histrelin implant, Palivizumab, Hyaluronic acid derivatives, Temozolomide, Antithrombin III (Human), Natalizumab, Panitumumab, Immune globulin (intravenous) (IVIG), Azacitidine, Verteporfin, Hyaluronidase, Bovine, Preservative Free, Naltrexone Depot, Teniposide, Omalizumab, 90Y-Ibritumomab tiuxetan, ADEPT, Aldesleukin, Alemtuzumab, Bevacizumab, Bortezomib, Cetuximab, Dasatinib, Erlotinib, Gefitinib, Gemtuzumab, Imatinib, Interferon alpha, Interleukin-2, Iodine 131 tositumomab, Lapatinib, Lenalidomide, Panitumumab, Rituximab, Sorafenib, Sunitinib, Thalidomide, Trastuzumab.

The auto-injector may also be used to deliver biologics or small molecule drugs including a wide range of medicinal products such as vaccines, blood and blood components, allergenics, somatic cells, gene therapy, tissues, and recombinant therapeutic proteins, and substances that are (nearly) identical to the body's own key signalling proteins may also be injected using the invention. Examples are the blood-production stimulating protein erythropoietin, or the growth-stimulating hormone named (simply) “growth hormone” or biosynthetic human insulin and its analogues.

The auto-injector may also be used to deliver monoclonal antibodies. These are similar to the antibodies that the human immune system uses to fight off bacteria and viruses, but they are “custom-designed” (using hybridoma technology or other methods) and can therefore be made specifically to counteract or block any given substance in the body, or to target any specific cell type.

The auto-injector may also be used to deliver receptor constructs (fusion proteins), usually based on a naturally-occurring receptor linked to the immunoglobulin frame. In this case, the receptor provides the construct with detailed specificity, whereas the immunoglobulin-structure imparts stability and other useful features in terms of pharmacology.

The auto-injector may also be used to deliver any of the following: Alpha1-Adrenergic Antagonists, Analgesic Agents, Anesthetics, Angiotensin Antagonists, Inflammatory Agents, Antiarrhythmics, Anticholinergics, Anticoagulants, Anticonvulsants, Antidiarrheal Agents, Antineoplastics and Antimetabolites, Antineoplastics and Antimetabolites, Antiplasticity Agents, Beta-Adrenergic Antagonists, Bisphosphonates, Bronchodilators, Cardiac Inotropes, Cardiovascular Agents Central Acting Alpha2-stimulants, Contrast Agents, Converting Enzyme Inhibitors, Dermatologics, Diuretics, Drugs for Erectile Dysfunction, Drugs of Abuse, Endothelin Antegonists, Hormonal Agents and Cytokines, Hypoglycemic Agents

Hypouricemic Agents and Drugs Used For Gout, Immunosuppressants, Lipid Lowering Agents, Psychotherapeutic Agents, Renin Inhibitors, Serotonergic Antagonist Steroids, Sympathomimetics, Thyroid and Antithyroid Agents, Vasodilators, Vasopeptidase Inhibitor.

The auto-injector may also be used to deliver any drug with indications for Rheumatoid arthritis or Multiple sclerosis, Hemophilia A or B, Vasculitis, Beta-thalassemia, Anemia, blood coagulation disorders, Von Willebrand disease, Sickle cell anemia, Solid Tumours, Leukemia, all cancers including liver, bladder, renal, esophageal, ovarian, breast, prostate, pancreatic, colorectal or lung, malignant melanoma, multiple myeloma, crohn's disease, ulcerative colitis, uveitisfabry disease, pompe disease, viral infections, HIV, Hepatitis A, B, C, Marburg virus, Wolman disease, Muscular dystrophy, botulism, muscular diseases, ebola virus, gout, acne, psoriasis, COPD, asthma, Alzheimer's, ALS, migraine, synovitis, fibrosis or any other indication.

The auto-injector may also be used to deliver any drug approved and listed by the FDA in the USA or any other national or international agency. Additionally any generic or biosimilar or biobetter drug on the market or in development.

The auto-injector may also be used to deliver any one of the following: Lipitor, a cholesterol-lowering statin drug, Nexium, an antacid drug, Plavix, a blood thinner, Advair, Abilify, an antipsychotic drug, Seroquel, an antipsychotic drug, Singulair, an asthma drug; Crestor, a cholesterol-lowering statin drug, Actos, a diabetes drug or Epogen, an injectable anemia drug.

The auto-injector may also be used to deliver any other drug not listed above capable of being injected and available at present or being developed by any pharmaceutical company or any other company anywhere in the world.

The invention may be used to inject humans or animals.

### SPECIFIC EMBODIMENTS OF THE INVENTION

Specific embodiments of the invention will now be described with reference to the figures in which:

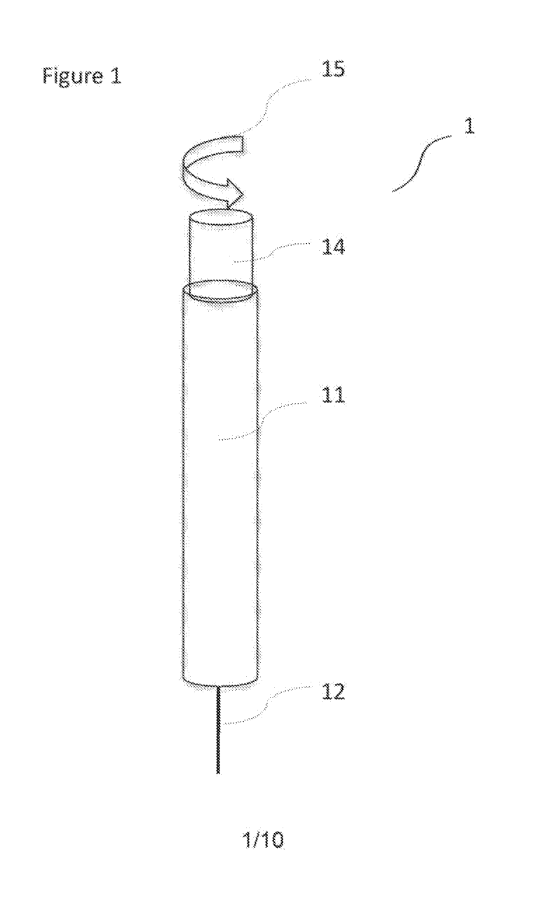

FIG. 1 is a schematic illustration showing an external view of an auto-injector according to the present invention.

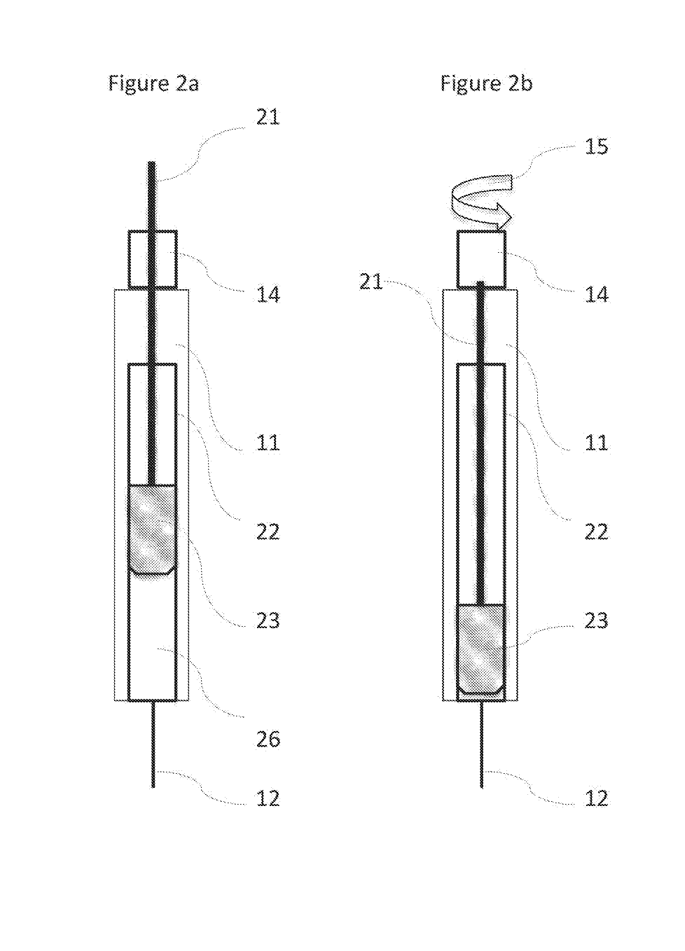

FIG. 2 shows a cross-sectional view of the auto-injector before (2a) and after (2b) injection.

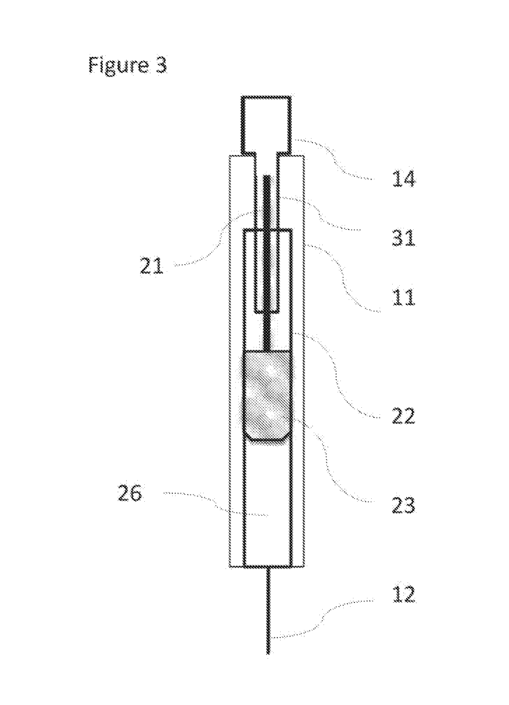

FIG. 3 shows a cross-sectional view of an alternative embodiment of an auto-injector according to the present invention.

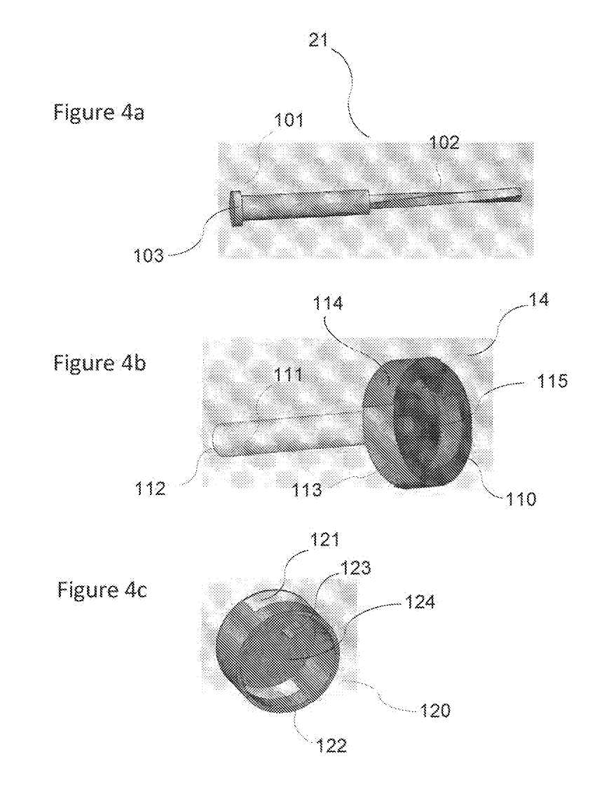

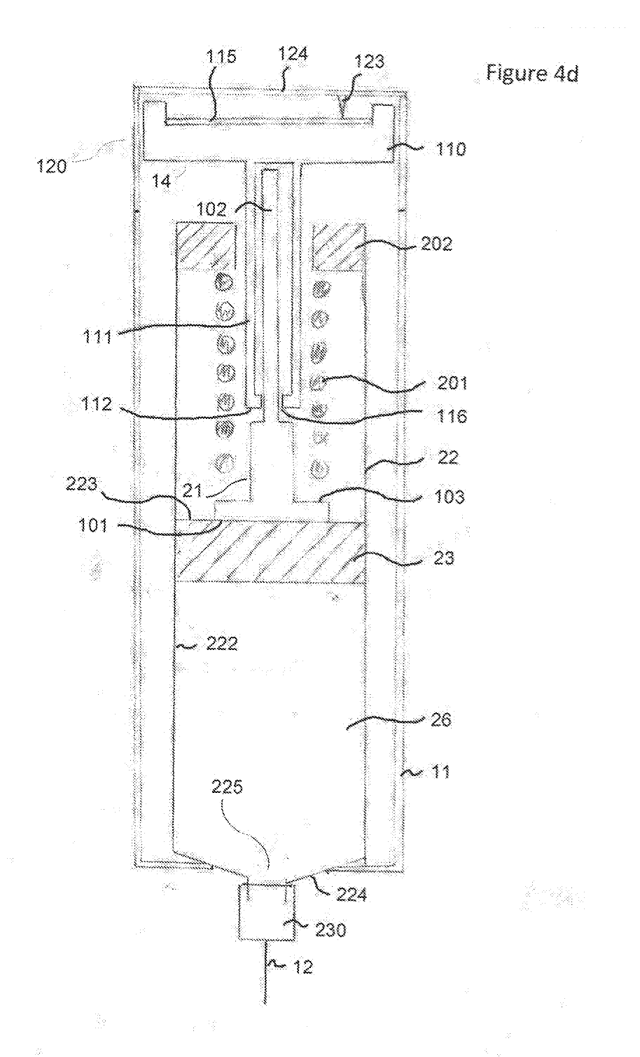

FIG. 4a is a schematic illustration of an indication member for an auto-injector according to the present invention.

FIG. 4b is a schematic illustration of a rotatable indicator for engagement with the indication member of FIG. 4a.

FIG. 4c is a schematic illustration of an end cap for an auto-injector according to the present invention.

FIG. 4d is a schematic cross-sectional illustration showing how the components of FIGS. 4a, 4b and 4c are assembled together.

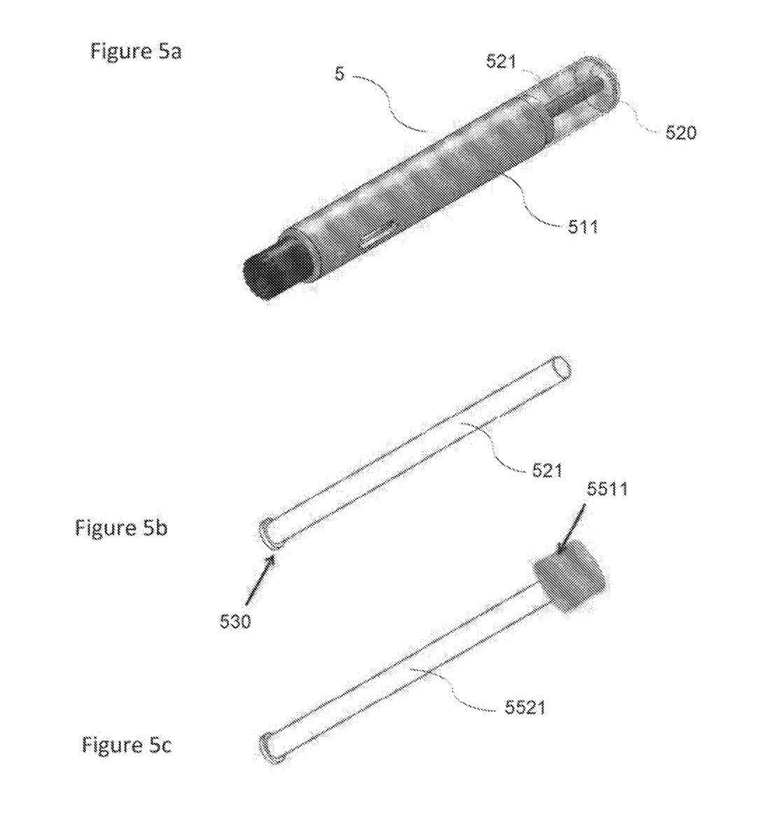

FIGS. 5a, 5b, and 5c are schematic illustrations of an alternative embodiment of an auto-injector according to the present invention.

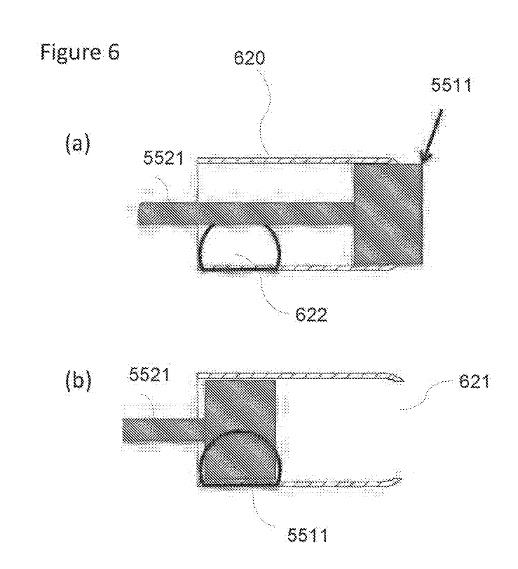

FIGS. 6a and 6b illustrate an embodiment of an end cap for an auto-injector designed to provide tactile feedback on the injection progress to a user of the auto-injector.

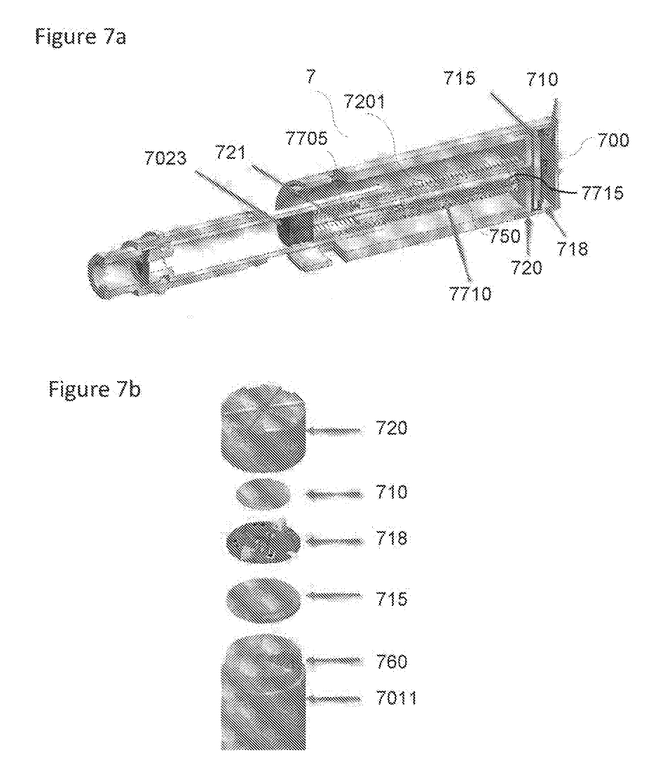

FIGS. 7a and 7b provide schematic illustrations of an embodiment of an auto-injector having indication means which provides audio feedback by electronic means.

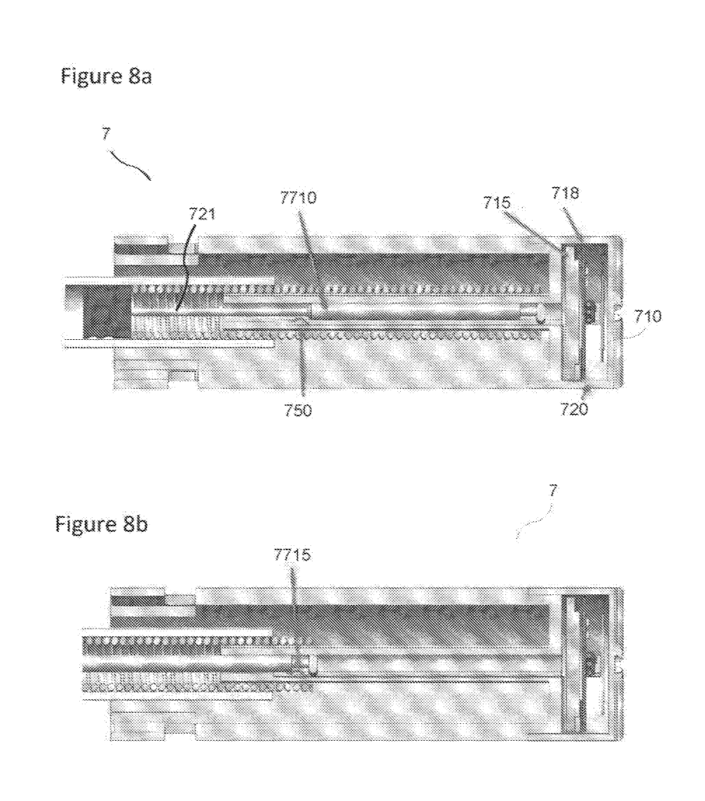

FIGS. 8a and 8b are cross-sectional views of the auto-injector of FIG. 7 before (8a) and after (8b) injection.

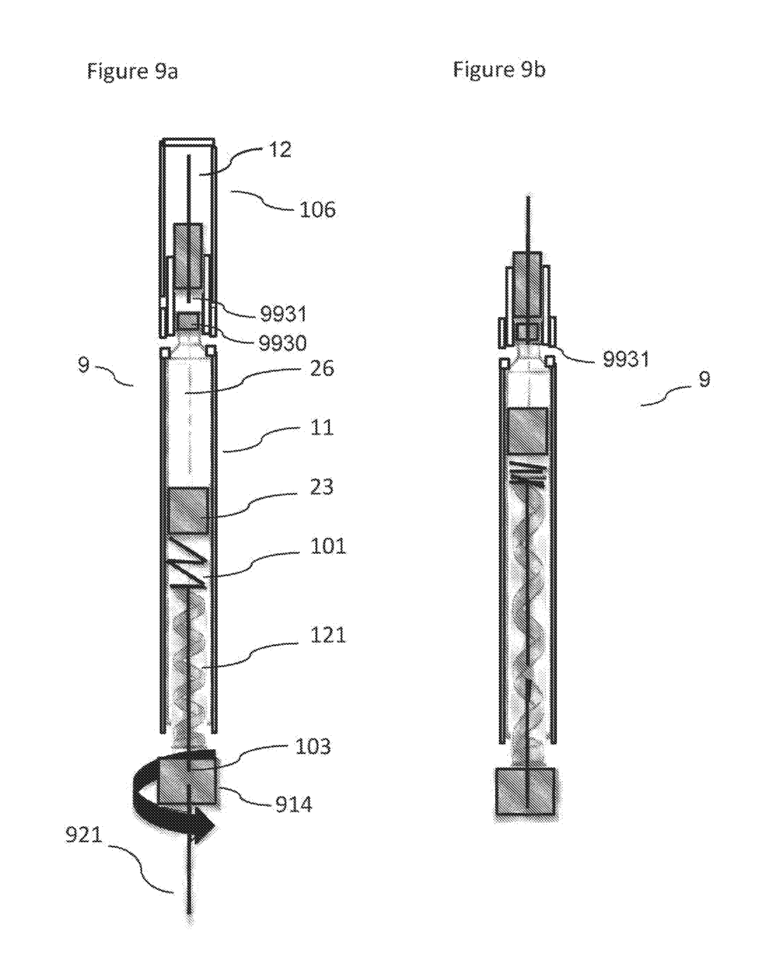

FIGS. 9a and 9b are schematic illustrations of an embodiment of an auto-injector, in which an elongate indication member is configured to release a second force upon rotation of a nut.

FIG. 1 provides a schematic illustration of an auto-injector for delivering a liquid medicament according to a specific embodiment of the invention. The auto-injector 1 has an external casing 11 and a rotatable indicator 14 projecting from a top end of the casing. A needle 12 for injection projects from a bottom end of the casing. During an injection, the indicator 14 rotates relative to the casing 11 as shown by arrow 15.

FIG. 2a and FIG. 2b are cross-sectional illustrations of the auto-injector 1 showing the components of the auto-injector 1 before use (FIG. 2a) and during injection (FIG. 2b). A glass or plastic cartridge 22 is located within the external casing 11. The cartridge 11 may alternatively be a syringe. A collapsible container containing a liquid medicament 26 is defined in part by internal walls of the cartridge and in part by a piston 23 that is slidably arranged within the cartridge 22. The piston 23 may alternatively be termed a plunger. An elongate indication member or rod 21 is attached to the piston 23 and engages with a rotatable indicator 14. A spring (not shown) is arranged within the cartridge and applies a biasing force to the piston that acts to pressurise the liquid medicament 26. A distal end of the cartridge is coupled to a needle 12 via a normally-closed valve (not shown). The normally-closed valve may be, for example, an aerosol valve, or may be a pierceable septum. When the normally-closed valve is opened the spring forces the piston 23 towards the distal end of the cartridge and the liquid medicament 26 is delivered out of the auto-injector via the needle 12. The rod 21 is coupled to the indicator 14 such that, when the rod 21 moves downwards with the piston 23, the indicator 14 rotates relative to the casing 11. In preferred embodiments the indication rod 21 may have a shaped cross-section, for example a flat rod having a rectangular cross-section, and the rod 21 may twist along its length. The rod 21 preferably engages with a rectangular slot in the indicator 14 to rotate the indicator. In other embodiments, the rod 21 may be cylindrical with a grooved thread that engages with a pin located in the indicator to rotate the indicator.

In FIG. 2b, the auto-injector has been actuated by opening the aerosol valve, and the liquid medicament has almost been delivered. The indication rod 21, being coupled to the piston 23, has traveled in a longitudinal direction towards the needle 12, thereby collapsing the collapsible container and delivering the liquid medicament 26. As the rod 21 travels, it passes through the indicator 14, thereby rotating it (in the direction of the arrow 15) relative to the casing 11.

FIG. 3 illustrates a further specific embodiment of an auto-injector. In the embodiment of FIG. 3, the indicator 14 has an internal extension 31 which engages with the indication rod 21 within the cartridge 22. This internal extension has the advantage that the rod 21 can be shorter, and therefore does not need to protrude from the casing 11 as in the embodiment of FIG. 2.

FIGS. 4a to 4d illustrate, in greater detail, an indication rod 21 and an indicator 14 for use in the auto-injector of FIG. 3. FIG. 4a illustrates the indication rod 21, FIG. 4b illustrates a rotatable indicator 14 for engagement with the indication rod 21, FIG. 4c illustrates an end cap for engaging with the rotatable indicator to provide visual and audible indication of injection progress, and FIG. 4d shows the position of the indicator on an assembled auto-injector.

FIG. 4a illustrates an example of an indication rod 21 suitable for use in conjunction with a rotatable indicator in an auto-injector, for example an auto-injector as described in relation to FIG. 3. The indication rod 21 has a distal end for engaging with a piston of an auto-injector, and a twisted proximal portion 102 located towards a proximal end of the rod 21. The twisted proximal portion 102 has a square-shaped cross-section that is twisted through at least 90 degrees along the length of the twisted proximal portion 102. The indication rod can advantageously be machined or moulded in a plastic material. A flange 103 is located at the distal end 101 of the indication rod 21. This flange helps to protect the piston from damage by the force applied by a spring.

FIG. 4b illustrates a rotatable indicator 14 for engagement with the indication rod 21 illustrated in FIG. 4a. The indicator 14 has a cylindrically-shaped rotatable head 110, and a cylindrical stem 111 depending from the rotatable head 110. The cylindrical stem forms the internal extension depicted in FIG. 3. The cylindrical stem is hollow, defining a space for accommodating the proximal portion 102 of the indication rod 21. The distal end 112 of the cylindrical stem 111 defines a square slot. This slot is shaped to engage with the square shaped cross-section of the twisted proximal portion 102 of the indication rod 21.

The cylindrical rotatable head 110 is of greater diameter than that of the cylindrical stem 111. A first section 113 of the edge of the rotatable head is coloured, for example in green. A second section 114 of the rotatable head is coloured in a contrasting colour, for example red. A ring of teeth 115, such as ratchet teeth, are machined into or formed in an upper surface of the rotatable head. The teeth 115 are configured to engage with a flexible projection such as a ratchet tang.

FIG. 4c shows an end cap 120 for engaging with an outer casing of an auto-injector and covering the rotatable head 110 of the rotatable indicator 14. Windows 121, 122 are defined through edges of the cap to allow the rotatable head 110 to be viewed. A flexible projection 123, which could be a ratchet tang, depends from an upper surface 124 of the cap for engagement with the teeth 115 of the rotatable head 110.

FIG. 4(d) is a schematic cross-sectional diagram showing the components of the indication means when assembled. As shown in FIG. 3, the auto-injector comprises a cartridge 22 positioned within an outer casing 11. The cartridge 22 defines a collapsible container containing a liquid medicament 26. The collapsible container is defined in part by internal walls 222 of the cartridge 22, and in part by a piston 23 that is slidably received within the cartridge 22. A helical spring 201 is located within the cartridge 22 by a stopper 202 and provides a biasing force that urges the piston 23 towards a distal end 224 of the cartridge 22. An opening 225 at the distal end 224 of the cartridge 22 is closed by a normally closed valve. A needle 12 is coupled to the normally-closed valve 230.

FIG. 4d shows how the indication rod 21 of FIG. 4a, the rotatable indicator 14 of FIG. 4b, and the end cap 120 of FIG. 4c engage with each other and with other components of the auto-injector. The distal end 101 of the indication rod 21 engages with an upper surface 223 of the piston 23. The helical spring engages with an upper surface of the flange 103 formed at the distal end 101 of the indication rod 21. As the biasing force from the spring is exerted on the piston 23 via the indication rod 21, the indication rod 21 is located in intimate contact with the piston. Thus, as the piston moves towards the distal end of the cartridge, so too does the indication rod move towards the distal end of the cartridge. A further advantage of supplying the spring force via a flange on the indication rod is that the piston, which may be formed from a resilient material such as an elastomer, is shielded to some degree from damage caused by interaction with the spring 201.

The twisted proximal portion 102 of the indication rod 21 extends through a slot 116 defined through the distal end 112 of the cylindrical stem 111. The proximal portion 102 is accommodated within the hollow body of the cylindrical stem 111.

The rotatable head 110 is connected to the cylindrical stem 111. The ring of teeth 115 are defined on an upper surface of the rotatable head 110 and engage with the flexible projection 123 that depends downwardly from the upper surface 124 of the end cap 120. The end cap 120 is fixed to an upper portion of the external casing 11.

In use, the normally closed valve 230 is opened to allow passage of the liquid medicament 26 from the cartridge 22. A biasing force applied by the spring between the stopper 202 and the piston 23 causes the piston to travel towards the distal end 224 of the cartridge 22, thereby delivering the liquid medicament. As the piston moves so does the indication rod. Engagement between the twisted proximal portion 102 of the indication rod 21 and the slot 116 of the rotatable indicator 14 causes the rotatable indicator to rotate. Progress of the injection can be visually determined by viewing the position of the rotatable head 110 through the windows 121, 122 defined through the end cap 120. As the rotatable head rotates, the flexible projection 123 is deflected by successive teeth 115. This creates a series of clicks, which provides an audible indication of the progress of injection.

FIG. 5(a) shows an auto-injector 5 according to an embodiment of the present invention. A transparent cap 520 fixed to an external casing 511 allows a user of the auto-injector 5 to see longitudinal movement of an indication rod 521 during injection. The indication rod 521 does not need to be twisted or otherwise incorporate means to translate longitudinal motion into rotational motion, and thus the auto-injector of FIG. 5 may be simpler to produce than, say, the auto-injector of FIG. 3.

FIG. 5(b) illustrates the indication rod 512 suitable for the auto-injector of FIG. 5a. The rod 521 is a cylindrical rod terminating at its distal end in a flange 530. As described above in relation to FIG. 4d this flange 530 acts to protect an auto-injector piston from damage caused by the force applied by the auto-injector spring.

FIG. 5(c) shows a further example of an indication rod 5521 suitable for use in an auto-injector of the type illustrated in FIG. 5a. The rod 5521 comprises a large, brightly-coloured indication portion 5511 at its proximal end, which increases the visibility of the indication rod.

FIGS. 6a and 6b show a portion of an auto-injector according to an embodiment of the invention which provides tactile feedback relating to the injection progress. The injector comprises an end cap 620 defining an end opening 621, through which an indication portion 5511 of an indication rod 5521 can project. The end cap 620 also defines a finger window 622, the finger window being an opening in a side wall of the cap 620 through which a user can feel movement of the indication rod 5521. As shown in FIG. 6(a), before injection, an indication portion 5511 of the indication rod 5521 protrudes from the end cap 620. A user of the auto-injector may be able to feel this protruding portion by touching it with a finger. However, the indicator portion would not be felt in the finger window prior to injection. At the end of the injection, as shown in FIG. 6(b), the indication portion 5511 has moved longitudinally towards the distal end of the auto-injector. The indicator portion no longer protrudes from the opening 621 in the end cap 620, but instead it can be felt through the finger window 622.

FIGS. 7a and 7b show an auto-injector 7 according to an embodiment of the present invention, which provides feedback on injection progress using electronic means. At a proximal end 700 of the auto-injector 7, a sounder 710 and a battery 715 are connected to a PCB 718. These components are located within an end cap 720, fixed to the proximal end of the auto-injector 700. Apart from the indication means, the components and operation of the auto-injector are as described above. Thus, only the indication means and its operation are described below.

As shown in FIG. 7(a), the auto-injector 700 comprises an elongate indication rod 721. The indication rod 721 comprises a central portion 7710 which has a wider diameter than the two end portions 7705, 7715. Electrical contacts 750 are positioned adjacent to the indication rod 721, and inside the main spring 7201 which pushes against the piston 7023 to pressurise a liquid medicament contained within the auto-injector 7.

FIG. 7(b) shows an exploded view of the components of the auto-injector that are located within the end cap 720. These components may be referred to as an electronics stack. The sounder 710, PCB 718, battery 715 and electrical contacts 760 are covered by and sit within the end cap 720 at the proximal end 700 of the auto-injector. The sounder 710 and battery 715 are connected to the PCB 718 by pressed metal spring contacts.

FIGS. 8a and 8b illustrate the indication means of the auto-injector 7 of FIG. 7. FIG. 8(a) shows the auto-injector 7 of FIG. 7 before injection, whereas FIG. 8(b) shows the auto-injector after injection. Before injection, the electrical contacts 750 are open, such that no current passes through the electrical circuit. During injection, the elongate indication rod 721 moves longitudinally towards a distal end of the cartridge, such that the wider central portion 7710 of the indication rod 721 closes the electrical contacts. This causes current to flow through the electrical circuit to make the sounder 710 produce audio feedback indicating the injection progress to a user of the auto-injector. At the end of injection, the narrower proximal end portion 7715 of the indication rod 721 reaches the electrical contacts 750, which allows the contacts to re-open. This stops the current flowing through the circuit, such that the sounder 710 is turned off, thus alerting the termination of injection to a user of the auto-injector.

FIGS. 9a and 9b show an auto-injector 9 according to an embodiment of the present invention.

FIG. 9a shows the auto-injector prior to injection, whereas FIG. 9b shows the auto-injector after injection. To prevent needle injuries and/or to maintain sterility, a needle shield 106 covers the needle 12 during storage of the auto-injector. This needle shield 106 is removed immediately prior to injection.

The auto-injector comprises a first helical spring 101 and a second helical spring 121, wherein the first spring 101 has a lower compression force than the second spring 121. The second spring 121 is held in a compressed state by a portion of an elongate indication rod 921 which hooks onto the second spring 121, in such away that the first spring 101 is compressed and provides a biasing force between the second spring 121 and the piston 23. This biasing force maintains the liquid contents 26 of the injector in a pressurized state, but at a lower pressure that would be generated by the second spring 121 were it to act directly on the piston 23. The elongate indication rod 921 comprises a screw thread 103 along at least a portion of its length which engages with a complimentary nut in an indicator portion 914. This engagement prevents longitudinal movement of the elongate indication rod prior to injection.

Rotation of the indicator portion 914 (e.g. by 45 to 360 degrees) releases the engagement between the screw thread 103 of the elongate indication member and the indicator portion 914, which in turn releases the second spring 121. Since the force applied by the second spring 121 is more powerful than the opposite force applied by the first spring 101, the elongate indication rod is pulled longitudinally towards a distal end of the auto-injector (see FIG. 9b). The second spring 121 compresses the first spring 101 and applies a force to the piston 23. The first spring 101 may be fully compressed. The second spring 121 thereby applies a force to the piston and to the collapsible container, which is larger than the force applied by the first spring 101 prior to release of the elongate indication member. This larger force urges the piston or stopper 23 towards a distal end of the medicament container and increases the pressure of the liquid medicament.

The auto-injector of FIG. 9 utilises a normally-closed valve in the form of a septum 9930 and a piercing needle In FIG. 9b, the piercing needle 9931 has perforated the septum 9930, thus allowing the liquid medicament to be delivered through the needle by the force applied to the collapsible container by the second spring 121.

1. An auto-injector for delivering a liquid medicament, wherein said auto-injector comprises:
a rigid outer container;
a collapsible container for containing the liquid medicament located within the rigid outer container, the collapsible container having a proximal end and a distal end,
an openable closure coupled to the distal end of the collapsible container for retaining the liquid medicament within the collapsible container;
an injection means coupled to the openable closure for delivering the liquid medicament from the collapsible container; and
pressurising means located within the rigid outer container, the pressurising means providing a force acting on the proximal end of the collapsible container that urges the proximal end of the collapsible container towards the distal end of the collapsible container, thereby pressurising the liquid medicament contained within the collapsible container such that the liquid medicament is delivered through the injection means when the openable closure is opened; the auto-injector further comprising:
an elongate indication member located at least partially within the rigid outer container and arranged to move longitudinally towards the distal end of the collapsible container when the openable closure is opened, in which longitudinal movement of the elongate indication member produces visual, and/or audio and/or tactile feedback relating to injection progress to a user of the auto-injector.

2. An auto-injector according to claim 1 in which the collapsible container is defined in part by rigid internal walls and in part by a piston that is slideably arranged within the rigid internal walls, such that the volume of the collapsible container varies depending on the position of the piston.

3. An auto-injector according to claim 2 in which the rigid internal walls of the collapsible container are defined by internal walls of the rigid outer container.

4. An auto-injector according to claim 2 in which the collapsible container is: a pharmaceutical glass or plastic cartridge, or a pre-filled syringe.

5. An auto-injector according to claim 1 in which the collapsible container is a bellows coupled to the openable closure.

6. An auto-injector according to any of the preceding claims in which a distal end of the elongate indication member is connected to a proximal end of the collapsible container.

7. An auto-injector according to any of the preceding claims in which the openable closure is a normally-closed valve, such as an aerosol valve.

8. An auto-injector according to any of the preceding claims in which the openable closure comprises a pierceable septum and a piercing means.

9. An auto-injector according to any of the preceding claims in which a portion of the elongate indication member extends beyond the rigid outer container, thereby providing a tactile and/or visual indication of injection progress to a user of the auto-injector.

10. An auto-injector according to any of the preceding claims in which the rigid outer container comprises a window through which a user of the auto-injector may monitor the movement of the elongate indication member, thereby providing an indication of injection progress to a user of the auto-injector.

11. An auto-injector according to any of the preceding claims in which said audio feedback is in the form of a series of clicks.

12. An auto-injector according to any of the preceding claims in which the elongate indication member is connected to an indicator portion, wherein longitudinal movement of the elongate indication member is translated into rotational movement of the indicator portion relative to the auto-injector casing and said rotational movement of the indicator portion provides the visual and/or audio and/or tactile feedback relating to the injection progress.

13. An auto-injector according to claim 12, wherein the elongate indication member has a shaped cross-section which twists along the length of a portion of the elongate indication member and the indicator portion has a slot shaped to engage with the elongate indication member such that longitudinal movement of the elongate indication member is translated into rotational movement of the indicator portion relative to the auto-injector casing.

14. An auto-injector according to claim 12, wherein the elongate indication member is cylindrical with a grooved thread and the indicator portion has a pin which is configured to engage with the grooved thread such that longitudinal movement of the elongate indication member is translated into rotational movement of the indicator portion relative to the auto-injector casing.

15. An auto-injector according to any of the preceding claims, wherein the elongate indication member provides visual, audio and/or tactile feedback by electronic means.

16. An auto-injector according to claim 15, wherein the elongate indication member comprises:
(a) a sounder which provides audio feedback upon movement of the elongate indication member; and/or
(b) a light, which provides visual feedback upon movement of the elongate indication member.

17. An auto-injector according to claim 15 or claim 16, wherein the elongate indication member is adapted to close electrical contacts as it moves longitudinally towards the distal end of the collapsible container, closure of the electrical contacts providing feedback relating to initiation and injection progress to a user of the auto-injector.

18. An auto-injector according to any of claims 15 to 17, wherein said elongate indication member comprises a battery and a printed circuit board (PCB).

19. An auto-injector according to claim 18, in which the elongate indication member comprises a sounder and in which the sounder and battery are connected to the PCB by pressed metal spring contacts.

20. An auto-injector according to any of the preceding claims in which the pressurising means is a biasing means, such as a spring.

21. An auto-injector according to any of the preceding claims in which the pressurising means is arranged to apply a first force to the collapsible container to continuously pressurise a liquid medicament contained therein during storage of the liquid medicament;
and wherein the pressurising means is actuatable to apply a second force to the collapsible container, the second force being greater than the first force.

22. An auto-injector according to claim 21, wherein the pressurising means is actuated to apply the second force to the collapsible container by depressing a button, or by removing a cap or a latch.

23. An auto-injector according to claim 22, wherein the elongate indication member comprises the latch.

24. An auto-injector according to claim 21 in which the elongate indication member comprises a screw thread and the auto-injector comprises a nut that is engageable with the screw thread,
wherein the pressurising means is actuated to apply the second force to the collapsible container by rotating the nut until the nut disengages the screw thread.

25. A method of using an auto-injector according to any of the preceding claims, the method comprising the steps of:
(a) opening the openable closure to allow delivery of the liquid medicament through the injection means and longitudinal movement of the elongate indication member towards the distal end of the collapsible container; and
(b) monitoring the injection progress via visual and/or audio and/or tactile feedback produced by the longitudinal movement of the elongate indication member.
